(19) United States
(12) Patent Application Publication
(10) Pub. No.: US 2025/0046430 A1
(43) Pub. Date: Feb. 6, 2025

(54) REGIONAL SEGMENTATION AND PARCELLATION OF THE HUMAN CEREBRAL CORTEX FROM COMPUTED TOMOGRAPHY

(71) Applicant: UNIVERSITY OF SOUTHERN CALIFORNIA, Los Angeles, CA (US)

(72) Inventors: Andrei Irimia, Los Angeles, CA (US); Nikhil Chaudhari, Los Angeles, CA (US)

(73) Assignee: UNIVERSITY OF SOUTHERN CALIFORNIA, Los Angeles, CA (US)

(21) Appl. No.: 18/794,766

(22) Filed: Aug. 5, 2024

Related U.S. Application Data
(60) Provisional application No. 63/530,881, filed on Aug. 4, 2023.

(51) Int. Cl. G16H 30/40; G06T 7/00; G06T 7/11; G16H 20/00

(52) U.S. Cl. CPC G16H 30/40; G06T 7/0014; G06T 7/11; G16H 20/00; G06T 2207/10081; G06T 2207/30016

(57) ABSTRACT

Systems, methods and devices are provided for evaluating changes in the human cerebral cortex from computed tomography. These may include regional segmentation and parcellation of the human cerebral cortex. Such a method may include receiving a plurality of brain images, segmenting aspects of the imaging, and performing other processing, such as correcting a radiodensity gradient, performing linear translations, and/or other steps. The system, methods, and devices may characterize brain volumes from the aggregated brain data. In this manner, changes in the brain may be determined.

### CROSS REFERENCE TO RELATED APPLICATIONS

This application is based upon and claims the benefit of and priority to U.S. Provisional Patent Application No. 63/530,881 entitled “REGIONAL SEGMENTATION AND PARCELLATION OF THE HUMAN CEREBRAL CORTEX FROM COMPUTED TOMOGRAPHY,” filed on Aug. 4, 2023, the entire content of which is incorporated by reference herein.

### STATEMENT AS TO FEDERALLY SPONSORED RESEARCH

This invention was made with government support under grant no. RF1 AG 054443, awarded by the (NIH) National Institutes of Health. The government has certain rights in the invention.

### BACKGROUND

### 1. Field

The following disclosure relates to medical imaging, and more specifically, to regional segmentation and parcellation from computed tomography imaging.

### 2. Description of the Related Art

Brain volume decreases with age. Monitoring the rate of brain volume change, and the location of significant areas of brain volume decrease, is critical for aging- and disease-related studies. Additionally, accelerated brain atrophy is associated with modern lifestyle factors such as high cholesterol, diabetes and hypertension. Comparison of the relationship between brain volume and age sheds light on the roles that lifestyle and environment play in determining the risk of developing dementia. However, monitoring the rate and location of brain volume change presents challenges for conventional imaging.

### SUMMARY

A method for analyzing age-related regional volumetric change of a brain is provided. The method may include segmenting, via a cortical parcellation scheme, cortical gray matter from a plurality of brain images into gyral and sulcal structures to form a gray matter probability map, the plurality of brain images generated from computed tomography (CT) scans of the brain. The method may include correcting a radiodensity gradient along an inferior-superior axis of a computed topography (CT) volume. The method may include iterating linear transformations to register the cortical gray matter to each of a subject's gray matter mask. The method may include generating an aggregated brain data for each subject's brain including a label for at least a set of voxels in the brain for each subject. The method may include normalizing brain volumes from the aggregated brain data for each subject.

In various embodiments, the method may include one or more further aspect(s). For instance, in various instances, the cortical parcellation scheme is a Destrieux cortical parcellation scheme. The method may include binarizing the gray matter probability map. The method may include enhancing registration quality of each of the subject's gray matter mask via non-linear registration. The method may include wherein the normalizing further includes dividing each regional volume of the subject's brain by a total intracranial volume of the subject's brain to form a set of normalized regional volumes for each subject's brain.

In various embodiments, the method may include analysis wherein the normalizing further includes performing a linear regression on the set of normalized regional volumes predicted by age for each subject's brain. The method may include computing standard regression coefficients by converting the set of normalized regional volumes for each subject's brain to z-scores prior to regression. The method may include comparing the set of normalized regional volumes of a first set of subject brains to the set of normalized regional volumes of a second set of subject brains. The method may include wherein the first set of subject brains and the second set of subject brains are determined based on a geographical location of each subject. The method may include calculating a rate of decrease of cortical gray matter volume of the first set of subject brains and the second set of subject brains. The comparing may further include comparing the rate of decrease of cortical gray matter volume of the first set of subject brains to the second set of subject brains. The comparing may further include comparing a portion of the first set of subject brains with a portion of the second set of subject brains based on a sex of each subject. The method may include associating a pattern of regional cortical atrophy in the first set of subject brains to lifestyle differences between each subject from the first set of subject brains relative to each subject in the second set of subject brains based on the comparing. In various embodiments, the method may include generating a recommendation of lifestyle changes for a subject in the second set of subject brains based on the pattern of regional cortical atrophy.

A method is provided. The method may include comparing a relationship between regional brain volumes and age of a first set of subjects to a second set of subjects. The method may include associating a pattern of regional cortical atrophy in the first set of subjects to subsistence lifestyle differences between each subject brain in the first set of subjects relative to each subject in the second set of subjects based on the comparing. The method may include generating a recommendation of lifestyle changes for a subject in the second set of subjects based on the pattern of regional cortical atrophy.

In various embodiments, the method includes normalizing brain volumes from brain images for each subject prior to the comparing, the brain images generated from computed tomography (CT) scans of the brain of each subject. In various embodiments, the normalizing further includes dividing each regional volume of the subject's brain by a total intracranial volume of the subject's brain to form a set of normalized regional volumes for each subject's brain. In various embodiments, the normalizing further includes performing a linear regression on the set of normalized regional volumes predicted by age for each subject's brain.

An article of manufacture is provided. The article may include a tangible, non-transitory computer-readable storage medium having instructions stored thereon that, in response to execution by one or more processors, cause the one or more processors to perform operations. The operations may include receiving, via the one or more processors, a plurality of brain images, the plurality of brain images generated from computed tomography (CT) scans of a brain of each subject. The operations may include segmenting, via the one or more processors and through a cortical parcellation scheme, cortical gray matter from the plurality of brain images into gyral and sulcal structures to form a gray matter probability map. The operations may include correcting, via the one or more processors, a radiodensity gradient along an inferior-superior axis of a computed topography (CT) volume.

The operations may include iterating, via the one or more processors, linear transformations to register the cortical gray matter to each of a subject's gray matter mask. The operations may include generating, via the one or more processors, an aggregated brain data for each subject's brain including a label for at least a set of voxels in the brain for each subject. The operations may include normalizing, via the one or more processors, brain volumes from the aggregated brain data for each subject.

In various embodiments, the operations may also include dividing each regional volume of the subject's brain by a total intracranial volume of the subject's brain to form a set of normalized regional volumes for each subject's brain and performing a linear regression on the set of normalized regional volumes predicted by age for each subject's brain.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the following illustrative figures. In the following figures, like reference numbers refer to similar elements and steps throughout the figures.

FIG. 1 illustrates a system for regional segmentation and parcellation of the human cortex from computed tomography, in accordance with various embodiments;

FIG. 2 illustrates a method for regional segmentation and parcellation of the human cortex from computed tomography, in accordance with various embodiments; and

FIG. 3 illustrates a further method for regional segmentation and parcellation of the human cortex from computed tomography, in accordance with various embodiments.

### DETAILED DESCRIPTION

A system, apparatus and/or method for regional segmentation and parcellation of the human cortex from computed tomography is disclosed herein. However, prior to discussing the system, helpful context is provided by including a detailed evaluating of an example implementation to showcase features, benefits, and aspects of the system, apparatus and/or method.

Brain volume decreases with age. Monitoring the rate of brain volume change, and the location of significant areas of brain volume decrease, is critical for aging- and disease-related studies. Additionally, accelerated brain atrophy is associated with modern lifestyle factors such as high cholesterol, diabetes and hypertension. Comparison of the relationship between brain volume and age between populations sheds light on the roles that lifestyle and environment play in determining the risk of developing dementia.

Brain volumetry allows the study of disease-related neurobiological mechanisms, enabling the monitoring of neurodegenerative diseases, e.g., Alzheimer's disease (AD). Previous studies have not compared age-related regional volumetric change. Computed tomography scans are segmented. Cortical gray matter (GM) is parceled into atlas regions. Sex-specific linear regression coefficients representing the annual change in regional volume are compared to a reference population.

The results rendered by the system, apparatus, and method for regional segmentation and parcellation of the human cortex from computed tomography indicate significantly different patterns of cortical atrophy between populations.

The disclosure herein includes a discussion comparing the rate of change in regional brain volumes in a study participant or group of study participants to a reference population. The discussion also includes a novel system, apparatus, and method including an algorithm to parcellate computed tomography (CT) scans to examine the effect of age and sex on regional brain volumes.

In addition to disclosing a novel system, apparatus, and method for regional segmentation and parcellation of the human cortex from computed tomography, this disclosure provides the first study to report a positive cross-sectional trend of regional brain volume with age ever reported in a human population. Because the measurement of genuine hypertrophy requires longitudinal measurements, the discussion refer to this trend as pseudo-hypertrophy, where the prefix pseudo implies that the study is cross-sectional.

A total of 1,180 Tsimane and Moseten and 19,973 participants in the UK Biobank (UKBB, https://www.ukbiobank.ac.uk/) participated in imaging according to regional segmentation and parcellation technology disclosed herein. For UKBB participants, T₁-weighted magnetic resonance imaging (MRI) scans were acquired at 3 T using Siemens Skyra MRI scanners (software platform VD13, 32-channel receiving head coil, 3D acquisition, magnetization-prepared rapid gradient-echo sequence, voxel size=1.0 mm×1.0 mm×1.0 mm, matrix size=208×256×256, inversion time [TI]=800 ms, repetition time [TR]=2 s, in-plane acceleration factor=2). For Tsimane and Moseten participants, CT scans were acquired using a 16-detector row scanner (General Electric BrightSpeed, Milwaukee, WI). Images were acquired clockwise, in helical mode, with a standard convolution kernel, and two reconstructions: one with a voxel size of 1.25 mm×1.25 mm×1.25 mm, and another with a voxel size of 0.625 mm×0.625 mm×0.625 mm. Additional parameters include a kilovoltage peak of 120 kV, a data collection diameter of 25 cm, a mean exposure time of 1.417 s, an X ray tube current of 140 mA, and a focal spot of 0.7 mm.

The discussion now shifts to an elaboration of image processing implemented in the system, apparatus, and method disclosed herein. In various embodiments, software is used to segment CT scans using a two-step approach. First, the brain was segmented. Second, an algorithm was used to segment cortical gray matter (GM) into gyral and sulcal structures according to the Destrieux parcellation scheme. The GM probability map was binarized. A spatial bias function was used to correct the radiodensity gradient along the inferior-superior axis of the CT volume. Next, three successive linear transformations (rigid, similarity, and affine) were applied iteratively to register the cortical GM of the atlas to each participant's GM mask. A non-linear registration was used to improve registration quality. The atlas is labeled according to a parcellation scheme, so this registration allows one to label all the voxels in the subject's brain according to the parcellation scheme.

Regarding CT/MRI validation, regional CT segmentations were validated using CT and MRI scans acquired from 13 U.S. adults aged 55 to 75. CT scans were acquired using a Toshiba, Aquilion ONE scanner and had scan parameters akin to those of Tsimane/Moseten scans. Images were acquired clockwise, in helical mode, with a Toshiba FC68 convolution kernel and a voxel size of 0.46 mm×0.46 mm×0.6 mm. Additional parameters included a kilovoltage peak of 120 kV, a data collection diameter of 32 cm, a mean exposure time of 1.000 s, an X-ray tube current of 140 mA, and a focal spot of 0.8 mm. T₁-weighted MRIs were acquired at 3 T using a Prisma MAGNETOM Trio TIM scanner (Siemens Corp., Erlangen, Germany) and a magnetization-prepared rapid acquisition gradient echo sequence was used with the following parameters: TR=1.950 s; echo time=3 ms; TI=900 ms; flip angle=9 degrees; percentage sampling=100; pixel bandwidth=240 Hz/pixel; matrix size—256×256; voxel size=1 mm×1 mm×1 mm.

Let r=1, . . . , R be a cortical structure in a cortical parcellation scheme, where R=148. Let wᵣ denote the percentage of total GM volume occupied by r. Let vᵢᵣ^{CT} be the volume of r for subject i, as derived from CT, and let vᵢᵣ^{MRI} be the volume of r for subject i, as derived from MRI. The average difference δvᵣ between vᵢᵣ^{CT} and vᵢᵣ^{MRI} was computed as:

$$δ {v}_{r} = \frac{1}{N} {\sum }_{i = 1}^{N} ( {v}_{ir}^{CT} - {v}_{ir}^{MRI} ) \qquad ( 0 )$$

where N is the total number of participants in the validation sample. The average of the weighted absolute differences, i.e.,

$$ϖ = \frac{1}{R} {\sum }_{r = 1}^{R} {w}_{r} \vert {δ}_{r} \vert$$

was used to measure the agreement between CT and MRI segmentations.

Regarding, age-related regional volume trajectories, to ease interpretation, two standardizations were applied to brain volumes. In the first of these, each regional volume was divided by total intracranial volume (TICV) to account for variation in head sizes. In the second standardization, a simple linear regression where the TICV normalized regional volume was predicted by age was performed. A second normalization adjusted regional volumes such that the volumes predicted by the best fitting regression line would start at 100 percent at the youngest age. Let β be the regression coefficient denoting the cross-sectional annual rate of change in regional volume, adjusted for head size and for the regressed mean volume at the youngest age of 46 years.

With these standardizations, a negative β indicates that regional volume decreases at a rate of β%/year relative to the initial age of 46, when regional volume is 100%. Regression coefficients describing how regional volumes trend with age were calculated for the Tsimane (β_{T}), Moseten (β_{M}), and UKBB (β_{UK}) participants. Standardized regression coefficients βₛ were computed by converting normalized brain volumes and ages to z-scores prior to regression, thereby facilitating direct comparison between groups' age-related effects on regional volume (negligible: β_{S}<0.10; small: 0.10<β_{S} 0.29; medium: 0.30<β_{S}<0.49; large: β_{S}>0.50).

Confidence intervals for β_{T}, β_{M} and β_{UK} were obtained through bootstrapping, which was implemented separately for males and females within each cohort. One thousand random subsamples of size 100 were drawn from the Tsimane or Moseten samples (male or female), and an age- and sex-matched subsample of size 100 was equivalently drawn from the UKBB sample. βs were calculated at every realization. After 1000 realizations, μ(β) and σ(β) were computed. To determine whether there was a significant effect of age on standardized brain volume, the null hypothesis H₀:β=0 was tested at a significance threshold α=0.05 for each region's β. Bonferroni corrections with α=0.05/148 were implemented for multiple comparisons.

Turning now to a comparison between Tsimane/Moseten and UKBB, Welch's two-tailed t-test for independent samples with unequal variances was used to test the null hypotheses H₀:β_{T}=β_{UK} and H₀:β_{M}=β_{UK} at a significance threshold α=0.05. Weights (wᵣ) of the cortical regions with significantly different regression coefficients between the Tsimane and UKBB were summed to give κ (the sum of all significantly different βs across all cortical regions). For calculation of κ if H₀:β₇=β_{UK} was rejected, and β_{T}<β_{UK}, then wᵣ was considered positive (i.e., regional volume loss was faster in the Tsimane compared to the UKBB). If H₀:β₇=β_{UK} was rejected, and β_{T}>β_{UK} then wᵣ was considered negative for calculation of κ (i.e., regional volume loss was faster in the UKBB compared to the Tsimane). The same was done to compare β_{M} to β_{UK}. In other words, if κ is positive, cortical GM volume is decreasing faster in Tsimane/Moseten compared to UKBB, and if K is negative, cortical GM volume is decreasing faster in the UKBB compared to Tsimane/Moseten.

Cognitive testing was also performed. A stick design test was used to assess visuospatial abilities in Tsimane and Moseten. This test includes reconstructing the printed designs of four models using four matchsticks. Variations in configuration, orientation of the whole figure and orientation of the matchsticks allow the test rater to score the test. For each group, a multiple linear regression was used to quantify the effects of standardized stick design scores (i.e., z scores of stick scores, zₛₛ) and standardized age (i.e. z scores of age, z_{Age}) on the total volume of regions with κ>1. Similarly to the estimation of β, variance estimates were obtained using 1000 bootstrapping iterations. The null hypothesis of no stick design score effect on age was tested.

The results are discussed in several paragraphs below. First, regarding quality assessment and validation of CT segmentation, segmentation quality was examined in 1,180 CT scans. Seven subjects were removed due to incorrigible segmentation errors. Of the remaining scans, 1,024 were selected to match the age range of the UKBB participants. In the validation set, ω̅ was found to be 2.5, indicating an average of 2.5% (where 0% indicates no volume difference) of MRI volumes were difference between the CT and MRI segmentations.

Regarding regional brain atrophy in Tsimane and Moseten, in Tsimane and Moseten, most (82%) regression coefficients β are negative, indicating cross-sectional decreases in regional cortical volume with age. Medium effect sizes (βₛ>0.3) are found in only X structures (˜2%). In Moseten males, 97 regions exhibit a negative trend of volume with age. Of these, the left planum polare of the superior temporal gyrus exhibits the largest effect size, decreasing at a rate of 1.38% per year (p<0.001, βₛ=−0.52). In Moseten females, 132 regions exhibit a negative trend of volume with age; of these, 37 have medium effect sizes. Similarly, in Tsimane males, 117 structures' volumes trend negatively with age; in Tsimane females, 139 do. In UKBB males, only the left superior frontal gyrus has a moderate effect size for β_{UK}, which trends negatively with age at a rate of −0.404% per year (p<0.001, βₛ=−0.34). In UKBB females, there are only small (βₛ<0.3) effect of age on regional volume decrease.

Regarding an increase in volume of occipital regions in Tsimane and Moseten, in Tsimane and Moseten, small but significant effects of pseudo-hypertrophy were found in occipital, posterior parietal, and posterior temporal structures, including the subparietal, medial occipito-temporal and lingual sulci. By comparison, in the UKBB cohort, all regions with β_{UK}>0 have negligible effect sizes (β_{S}<0.1). In Moseten males, 51 structures exhibit pseudo-hypertrophy, of which 24 have βₛ>0.1 (Table 3). In Moseten females, 16 structures exhibit this phenomenon and two of these have β_{S}>0.1. Similarly, in Tsimane males, there are 31 pseudo-hypertrophic structures (5 with β_{S}>0.1). In Tsimane females, there are 9 structures, two with βₛ>0.1. In Tsimane females, the inclusion of zss^{s} significantly decreases the βₛ pertaining to the total volume of pseudo-hypertrophic structures. Thus, βₛ=0.21 decreases to βₛ=0.19 after accounting for the effect of zₛₛ(t₃=−5.36, p=0.013). In Tsimane males, βₛ increases significantly with the inclusion of zₛₛ in the model, i.e., βₛ=0.19 increases to βₛ=0.22 after including z_{sS}(t₃=7.46, p=0.005). No significant changes in βₛ are observed in Moseten when the effect of cognitive score is included in regression.

The discussion now continues with a comparison of Tsimane/Moseten to UKBB participants. In the UKBB, the horizontal ramus of the anterior right lateral sulcus atrophies fastest in males; the right angular gyrus atrophies fastest in females. By comparison, in Tsimane and Moseten males, many frontal and temporal structures, including the right orbital sulci and collateral sulcus, atrophy at a significantly slower rate than in UKBB males. In Tsimane and Moseten females, the right sub-parietal sulcus atrophies significantly slower than the UKBB females.

As for findings in males, cortical GM of UKBB males atrophies faster than in Tsimane or Moseten. Of 137 structures whose trend with age differs significantly between UKBB and Tsimane, 54% decrease faster in the UKBB (κ=−17.08). Fifty-six percent of the 139 brain regions showing significant differences in β between UKBB and Moseten are decreasing faster in the UKBB (κ=−11.82). Regions that atrophy faster in the UKBB than in Moseten include frontal structures (e.g., the left vertical ramus of the anterior segment of the lateral sulcus and temporal structures (e.g., the medial occipito-temporal and lingual sulci).

As for findings in females, the cortical GM of Tsimane and Moseten females atrophies faster than UKBB females'. For 140 regions, the rate of volumetric change differs significantly between Tsimane and UKBB (κ=69.90). Of these 140, 87% decrease faster in Tsimane than in the UKBB. The left sub-parietal sulcus in particular is decreasing significantly faster in Tsimane females. The rate of volumetric change of 141 brain regions in Moseten females are significantly different to those of UKBB females, and 85% of these are decreasing faster in the Moseten compared to the UKBB (κ=73.02). Structures that atrophy faster in the UKBB than in Moseten include parietal structures such as the left intraparietal and transverse parietal sulci.

Regarding, pseudo-hypertrophy, brain volume typically decreases with age, due to cell death, loss of synapses and cell volume, as well as due to decrease in dendritic arborization. However, in Tsimane and Moseten, occipital and parietal regions often implicated in navigation, visual processing, and naming exhibit small pseudo-hypertrophy with age. In Tsimane, parieto-occipital pseudo-hypertrophy is partially explained by visuospatial performance. In Tsimane females, visuospatial ability partially explains pseudo-hypertrophy of the left subparietal and left posterior lateral sulcus such that it lessens effect of ages on regional volumes. In Tsimane males, visuospatial ability partially explains parieto-occipital pseudo-hypertrophy such that it increases the effect of ages on regional volumes.

Compared to other lobes, the occipital lobe is relatively preserved in industrialized populations. Nevertheless, there may be no other report of occipital volume (pseudo) hypertrophy. In indigenous Australians, the volume of visual cortex is preserved in comparison to Caucasian Australians, potentially reflecting the former's adaptation to living in forests and deserts. The same may be the case in Tsimane and Moseten, who live in densely forested areas where they rely on complex visual cues to navigate and subsist. In support of this hypothesis, structures such as the subparietal sulcus—which is involved in memory recall, visual scene processing, and navigation—exhibit pseudo-hypertrophy in Tsimane and Moseten.

The regional pseudo-hypertrophy reported here may be due to high levels of physical activity. Studies of older adults in industrialized countries such as the US suggest that physical activity can decelerate brain atrophy and perhaps even lead to regional increases in cortical GM. For example, in older adults, one study reported that six months of aerobic fitness training was associated with increases in GM and white matter volume. Aerobic exercise leads to the production of growth hormones such as brain-derived neurotrophic factor and insulin-like growth factor. These hormones facilitate the creation of capillaries, the synthesis of dendritic connections, and the birth of cells in the hippocampus.

It may also be useful to compare Tsimane/Moseten data to UKBB data. In Tsimane, the cross-sectional rate of brain volume decrease with age is −0.21% per year, which is significantly slower than in the US and EU. As reported here, this rate is not the same across the brain, nor is it the same across sexes or populations. Instead, in Tsimane and Moseten compared to the UK, some temporal, frontal and parietal regions trend more weakly with age or even and experience hypertrophy. In Tsimane and Moseten, X % of the cortex has a rate of brain volume decrease with age that is more negative than that of persons in the UK. Furthermore, Tsimane and Moseten females' total cortical GM trends negatively with age faster than in sex- and age-matched individuals in the UK.

Regional differences in the trend of brain volume with age may be due to sex differences in sex hormones, lifestyle, or health trajectories. In industrialized populations, the frontal and temporal lobes, striatum, cerebellum, and hippocampus atrophy faster than the rest of the brain. In males, frontal and temporal regions atrophy fastest, while in females the hippocampus and parietal lobes atrophy fastest.

Tsimane and Moseten males exhibit a weaker dependence of total brain volume on age compared to samples in the UK and EU. Similarly, males' regional GM volume pseudo-atrophies slower with age than in UK males. By contrast, Tsimane and Moseten females exhibit faster pseudoatrophy compared to UK females. The majority (85%) of structures pseudo-atrophy faster in Moseten females compared to UK females, while only half as many regions atrophy faster in Moseten males compared to UK males. The reason for such a stark contrast between Tsimane and Moseten females, on the one hand, and UK females, on the other hand, is unclear. In principle, the protective effect of female sex hormones on health is reversed after menopause. However, it is unclear why this might affect Tsimane and Moseten females more than females in the UK.

As for regions atrophying fastest in Tsimane/Moseten, this trend is likely due to phenomena such as gyral shrinking and sulcal widening, which have been documented thoroughly in industrialized populations. Expectedly, in Tsimane and Moseten, the inferior frontal gyri atrophy faster than other structures. Faster frontal atrophy may reflect diminished higher-order cognitive functions such as semantic and working memory, impulse control and inhibition, speech production and phonological processing, planning, and sensory integration.

Like in industrialized populations, it was found that the left superior temporal gyrus and the bilateral anterior transverse temporal gyrus atrophy faster in the Tsimane and Moseten. The left superior temporal gyrus, which is longer than the right superior temporal gyrus, is involved in speech perception and production. Finally, the short insular gyri atrophy faster than other regions in the Tsimane and Moseten. The insulae are linked to audio-visual integration tasks, consciousness, emotion regulation, and homeostasis. As more cognitive data for the Tsimane and Moseten becomes available, the relationship between regional brain volume changes and cognitive functions can be further explored.

Brain atrophy, in this cross-sectional design, is estimated as annual change in regional volume and not directly measured within participants. Although extremely valuable longitudinal studies of aging exist, only a small part of the five-decade age span sampled in this cross-sectional investigation could be covered in a longitudinal study. It is well established that brain atrophy increases after age of 70, however few Tsimane and Moseten adults who are older than the age of 70 are alive. Comparisons between industrialized and non-industrialized populations after the sixth decade would reveal further information about age-related brain volume loss as illnesses such as dementia become more apparent. Finally, CT rather than MRI was the only imaging method available for the Tsimane and Moseten samples. This study provides a new method of segmenting CT scans, allowing for the comparison with MRI.

The Tsimane and Moseten forager-horticulturalists of Bolivia exhibit discrete patterns of atrophy compared to individuals living in an industrialized society. Where the UK population, on average, atrophy faster in frontal and temporal regions, similar brain regions in the Tsimane and Moseten are significantly more preserved with age. The Tsimane and Moseten females show faster atrophy when compared to their industrialized counterparts; however, the males are relatively preserved. The Tsimane and Moseten also exhibit age-related increases in volume in brain regions important for visuospatial abilities and cognitive abilities that are integral to their survival in the Amazonian forests.

Having introduced an example use scenario for the system, method, and apparatus disclosed herein, further details regarding an example implementation are provided. For instance, to perform tissue classification, voxel intensity values are used to assign their probabilities of belonging to one of several tissue classes by estimating the parameters of the intensity distributions of each class. This is accomplished by first defining an objective function derived from a mixture of Gaussian random variable models, and by then minimizing the value of this function using a parameter optimization process. A set of a priori tissue probability maps specified in a standard space (atlas) are used to assist the classification. The objective function can assist this process by weighing the probability maps of the standard space according to Bayesian inference principles and then deforming them so that they match the volumes being segmented. Specifically, the template is warped to each subject's brain volume, after which the latter can be segmented and the ensuing spatial classifications can be smoothed. When combined with a priori information specified by the template, Bayesian inference can be used to calculate posterior probabilities, based on each subject's voxel intensity values. The interface between the resulting grey matter (GM) and white matter (WM) volumes is smoothed according to principles inspired from nonlinear filter theory, subject to topological constraints dictated by the structural neuroanatomy of the human brain.

The distribution of image intensities in a neuroimaging volume is modeled here by a mixture of K clusters, each including Gaussian random variables. Each Gaussian variable is parametrized by its mean μₖ, variance ok and mixing coefficient γₖ, subject to the constraint that the sum of all mixing coefficients must be equal to 1. Fitting this Gaussian mixture model to the image intensity data vector y of length l involves maximizing the probability of observing the data given the model parametrization. The probability that a voxel has intensity yᵢ given that it belongs to the k-th Gaussian random variable (i.e., given that cᵢ=k) parametrized by μₖ and σₖ² is

$$P ( {y}_{i} \vert {c}_{i} = k , {μ}_{k} , {σ}_{k}^{2} ) = \frac{1}{{( 2 {πσ}_{k}^{2} )}^{1 / 2}} exp [ - \frac{{( {y}_{i} - {μ}_{k} )}^{2}}{2 {σ}_{k}^{2}} ] . \qquad ( 1 )$$

Because the probability that yᵢ belongs to the k-th Gaussian random variable given the proportion γₖ of voxels which belong to that random variable is P(cᵢ=k|γₖ), Bayes' rule indicates that

$$P ( {y}_{i} \vert {c}_{i} = k , {μ}_{k} , {σ}_{k}^{2} , {γ}_{k} ) = P ( {y}_{i} \vert {c}_{i} = k , {μ}_{k} , {σ}_{k}^{2} ) P ( {c}_{i} = k \vert {γ}_{k} ) , \qquad ( 2 )$$

and the total probability of observing yᵢ becomes

$$P ( {y}_{i} \vert μ , σ , γ ) = {\sum }_{k = 1}^{K} P ( {y}_{i} , {c}_{i} = k \vert {μ}_{k} , {σ}_{k}^{2} , {γ}_{k} ) , \qquad ( 3 )$$

whilst the probability

$$P ( y \vert μ , σ , γ ) = {\prod }_{i = 1}^{I} P ( {y}_{i} \vert μ , σ , γ ) \qquad ( 4 )$$

that all I intensities in y are observed given μ, σ and γ can be maximized by varying the latter parameters in the cost function

$$ε = - log P ( y \vert μ , σ , γ ) . \qquad ( 5 )$$

Regarding spatial priors, deformation and regularization, a probabilistic atlas is used to specify the prior probability that each voxel belongs to any tissue class in the Gaussian mixture model. This is done without assuming that any intensity distribution for each class is Gaussian, such that the prior probability of voxel i being drawn from the k-th Gaussian distribution is

$$P ( {c}_{i} = k \vert γ ) = \frac{{γ}_{k} {P}_{ik}}{{\sum }_{j = 1}^{K} {γ}_{j} {P}_{ij}} , \qquad ( 6 )$$

where Pᵢₖ is the tissue probability for class k at voxel i. For voxels located at the boundary between tissues (e.g. the GM/WM boundary), this model accommodates the difficulty of ascertaining the class to which voxel i belongs. The atlas used here is based on an average of CT volumes. The original atlas has a resolution of 1×1×1 mm and its image intensities range from 0 to 90 in increments of 1.3×10⁻³.

Let α be a vector of diffeomorphic deformation parameters which allow the co-registration of the spatial template and a subject volume. Here, spatial priors are deformed according to α, to allow co-registration according to

$$P ( {c}_{i} = k \vert γ , α ) = \frac{{γ}_{k} {P}_{ik} ( α )}{{\sum }_{j = 1}^{K} {γ}_{j} {P}_{ij} ( α )} \qquad ( 7 )$$

With this adjustment, one obtains

$$ε = - {\sum }_{i = 1}^{I} log P ( y \vert μ , σ , γ , α ) \qquad ( 8 )$$

Or, more explicitly,

$$ε = - {\sum }_{i = 1}^{I} log {\sum }_{j = 1}^{K} P ( {c}_{i} = k \vert γ , α ) P ( {y}_{i} \vert {c}_{i} = k , {μ}_{k} , {σ}_{k}^{2} ) . \qquad ( 9 )$$

The parametrization of the deformation is implemented using a linear combination of sinusoidal transform bases subject to spatial regularization by maximizing P(y, α|μ, σ, γ). Only the lowest frequencies of a discrete sine transform were used, resulting in 392 (7×3×8) parameters to describe deformations along each spatial dimension. Three additional parameters were used to model linear scaling and one parameter was used to model linear image intensity inhomogeneities. The probability densities of the spatial parameters α are modeled by multivariate Gaussian random variables with mean 0 and covariance matrices C_{α}. The spatial regularization involving these covariance matrices and deformations prevents undesirable interactions between parameter estimates. Initially, parameter value estimates are assigned randomly, and nonlinear deformation coefficients are set to zero. Model parameters are then optimized using an expectation maximization (EM) algorithm, where the Gaussian mixture and deformations are re-calculated by iteratively updating exactly one while the others are held constant. Deformations are optimized using a Gauss-Newton scheme.

Regarding topology-constrained refinement, after probabilistic assignment of voxels to one of three classes (WM, GM or CSF), the segmentation is refined iteratively using a priori information concerning the local properties of the cortex. Specifically, because the surface defined by the WM/GM interface is smooth and its curvature is both defined and finite everywhere on it, the local topology of the brain can be used to correct the probabilistic tissue classification. This process is analogous to the application of a nonlinear, anisotropic filter whose nonlinearity is high near the WM/GM boundary. As the distance from some given voxel to the WM/GM boundary increases, the filter becomes more linear; because the true boundary is topologically smooth, the filter shape must be planar at this interface.

In this approach, the segmentation is corrected in two steps. First, one may identify the plane crossing the boundary which is intersected by voxels whose intensity variance is minimal. Once this is done, the voxels within this plane are examined to determine whether (A) a substantial proportion of them have ambiguous classifications based on their intensity or whether (B) they are surrounded by voxels whose class memberships vary greatly. If changing the class assignment of these voxels decreases the in-plane intensity variance, the voxels in question are re-assigned to their more appropriate class.

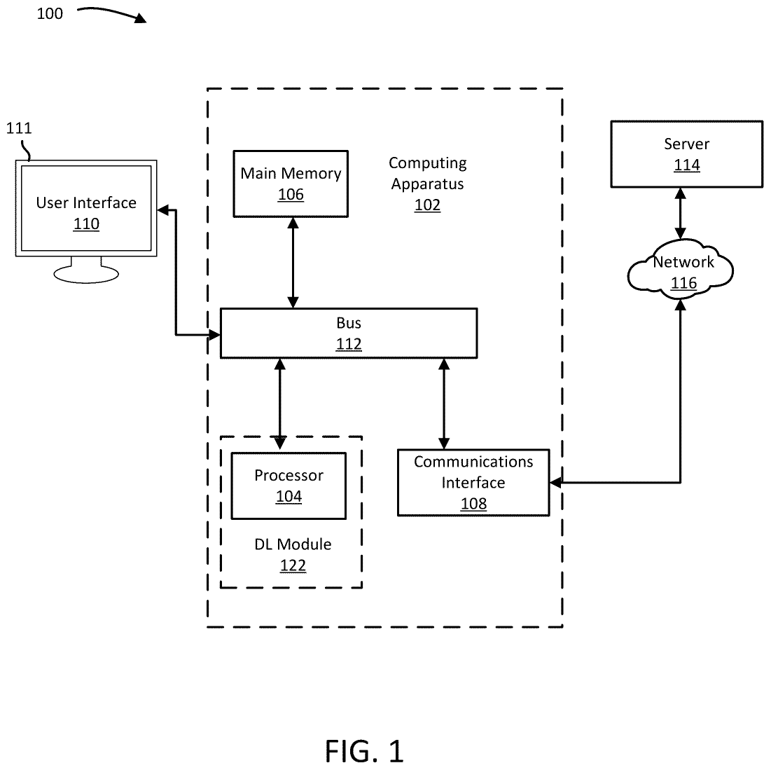

This disclosure provides one or more system, method, and apparatus for evaluating these brain features. Specifically, and referring now to FIG. 1, a system 100 (e.g., a computing system) for regional segmentation and parcellation of the human cortex from computed tomography is illustrated. The system 100 (e.g., a computing system) may include a computing apparatus 102. The computing apparatus 102 may include one or more processors 104, a memory 106 and/or a bus 112 and/or other mechanisms for communicating between the one or more processors 104. The system 100 may be a cloud computing system including one or more processors, servers, storage, databases, networking, software, analytics, and/or intelligence accessed or performed over or using the Internet (“the cloud”). The one or more processors 104 may be implemented as a single processor or as multiple processors. The one or more processors 104 may execute instructions stored in the memory 106 to implement the applications of the system 100.

The one or more processors 104 may be coupled to the memory 106. The memory 106 may include one or more of a Random Access Memory (RAM) or other volatile or non-volatile memory. The memory 106 may be a non-transitory memory or a data storage device, such as a hard disk drive, a solid-state disk drive, a hybrid disk drive, or other appropriate data storage, and may further store machine-readable instructions, which may be loaded and executed by the one or more processors 104.

The memory 106 may include one or more of random-access memory (“RAM”), static memory, cache, flash memory and any other suitable type of storage device or computer readable storage medium, which is used for storing instructions to be executed by the one or more processors 104. The storage device or the computer readable storage medium may be a read only memory (“ROM”), flash memory, and/or memory card, which may be coupled to a bus 112 or other communication mechanism. The storage device may be a mass storage device, such as a magnetic disk, optical disk, and/or flash disk that may be directly or indirectly, temporarily, or semi-permanently coupled to the bus 112 or other communication mechanism and be electrically coupled to some or all of the other components within the system 100 including the memory 106, the user interface 110 and/or the communication interface 108 via the bus 112. In various embodiments, the user interface 110 is configured to be displayed on a display device 111 (e.g., a monitor, a tablet, a phone, etc.).

The term “computer-readable medium” is used to define any medium that can store and provide instructions and other data to a processor, particularly where the instructions are to be executed by a processor and/or other peripheral of the processing system. Such medium can include non-volatile storage, volatile storage, and transmission media. Non-volatile storage may be embodied on media such as optical or magnetic disks. Storage may be provided locally and in physical proximity to a processor or remotely, typically by use of network connection. Non-volatile storage may be removable from computing system, as in storage or memory cards or sticks that can be easily connected or disconnected from a computer using a standard interface.

The system 100 may include a user interface 110. The user interface 110 may include an input/output device (e.g., a display device 111). The input/output device may receive user input, such as a user interface element, hand-held controller that provides tactile/proprioceptive feedback, a button, a dial, a microphone, a keyboard, or a touch screen, and/or provides output, such as a display, a speaker, an audio and/or visual indicator, or a refreshable braille display. The display device 111 may be a computer display, a tablet display, a mobile phone display, an augmented reality display or a virtual reality headset. The display device 111 may output or provide a data related to regional segmentation and parcellation of the human cortex from computed tomography.

The user interface 110 may include an input/output device that receives user input, such as a user interface element, a button, a dial, a microphone, a keyboard, or a touch screen, and/or provides output, such as a display, a speaker, headphones, an audio and/or visual indicator, a device that provides tactile/proprioceptive feedback or a refreshable braille display. The speaker may be used to output audio associated with the audio conference and/or the video conference. The user interface 110 may receive user input that may include configuration settings for one or more user preferences, such as a selection of joining an audio conference or a video conference when both options are available, for example.

The system 100 may have a network 116 connected to a server 114. The network 116 may be a local area network (LAN), a wide area network (WAN), a cellular network, the Internet, or combination thereof, that connects, couples and/or otherwise communicates between the various components of the system 100 with the server 114. The server 114 may be a remote computing device or system that includes a memory, a processor and/or a network access device coupled together via a bus. The server 114 may be a computer in a network that is used to provide services, such as accessing files or sharing peripherals, to other computers in the network.

The system 100 may include a communication interface 108, such as a network access device. The communication interface 108 may include a communication port or channel, such as one or more of a Dedicated Short-Range Communication (DSRC) unit, a Wi-Fi unit, a Bluetooth® unit, a radio frequency identification (RFID) tag or reader, or a cellular network unit for accessing a cellular network (such as 3G, 4G or 5G). The communication interface may transmit data to and receive data from the different components.

The server 114 may include a database. A database is any collection of pieces of information that is organized for search and retrieval, such as by a computer, and the database may be organized in tables, schemas, queries, reports, or any other data structures. A database may use any number of database management systems. The information may include real-time information, periodically updated information, or user-inputted information.

In various embodiments, the computing apparatus 102 can include a deep learning (“DL”) module 122. The DL module 122 can include the one or more processors 104. Stated another way, the DL module 122 can be run, or operated by the one or more processors 104. In various embodiments, the DL module 122 can perform the steps of the methods claimed herein and output an estimated genotype to the user interface 110. In various embodiments, the DL module 122 can be trained based on regional segmentation and parcellation of the human cortex from computed tomography for various populations. In various embodiments, based on the training, the DL module 122 can be configured to provide recommendations for lifestyle changes based on comparing regional volume data differences between relevant populations.

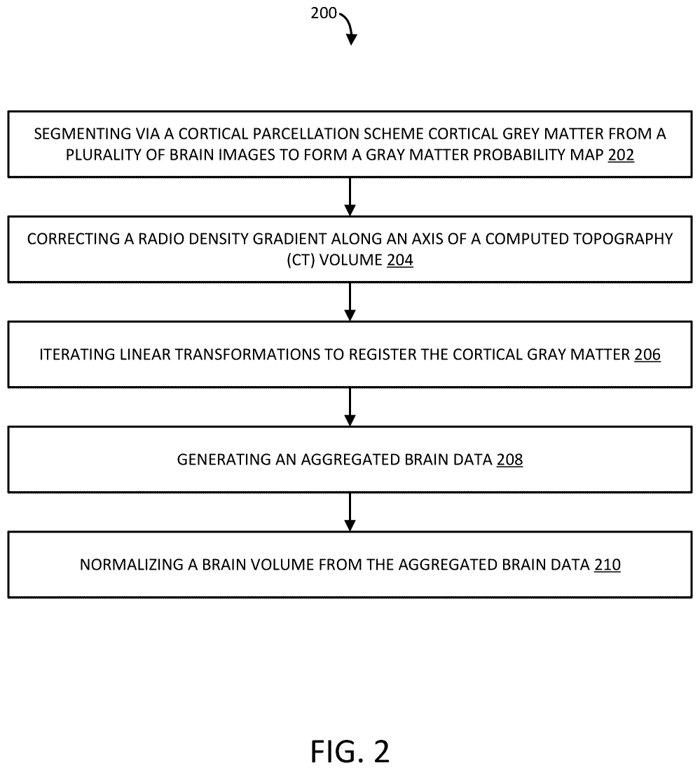

Having introduced both technical mechanisms and an associated computing system, it is now convenient to explain some example embodiments, in which various methods may be practically implemented in systems. For example, with reference to FIG. 2, an example method 200 may include a method for analyzing age-related regional volumetric change of a brain. The method 200 may include one or more further aspect. For instance, the method 200 may include segmenting, via a cortical parcellation scheme, cortical gray matter from a plurality of brain images into gyral and sulcal structures to form a gray matter probability map (block 202). The plurality of brain images may be generated from magnetic resonance imaging (MRI) scans of the brain. The method may include correcting a radiodensity gradient along an inferior-superior axis of a computed topography (CT) volume (block 204). The method may include iterating linear transformations to register the cortical gray matter to each of a subject's gray matter mask (block 206). The method may include generating an aggregated brain data for the brain including a label for at least a set of voxels in the brain for each subject according to the cortical parcellation scheme (block 208). The method may include normalizing the brain volume from the aggregated brain data (block 210).

In various embodiments, the cortical parcellation scheme is a cortical parcellation scheme. Moreover, in various embodiments, the method includes binarizing the gray matter probability map. The method may include enhancing registration quality of each of the subject's gray matter mask via non-linear registration. In various instances, the normalizing further comprises dividing each regional volume of the subject's brain by a total intracranial volume of the subject's brain to form a set of normalized regional volumes for each subject's brain. The normalizing may include performing a linear regression on the set of normalized regional volumes predicted by age for each subject's brain. The method may include computing standard regression coefficients by converting the set of normalized regional volumes for each subject's brain to z-scores prior to regression. The method may include comparing the set of normalized regional volumes of a first set of subject brains to the set of normalized regional volumes of a second set of subject brains. The first set of subject brains and the second set of subject brains are determined based on a geographical location of each subject. In various instances, the method includes calculating a rate of decrease of cortical gray matter volume of the first set of subject brains and the second set of subject brains. In various instances the comparing includes comparing the rate of decrease of cortical gray matter volume of the first set of subject brains to the second set of subject brains. In various instances, the comparing comprises comparing a portion of the first set of subject brains with a portion of the second set of subject brains based on a sex of each subject. The method may include associating a pattern of regional cortical atrophy in the first set of subject brains to subsistence lifestyle differences between each subject from the first set of subject brains relative to each subject in the second set of subject brains based on the comparing. Moreover, the method may include a recommendation of lifestyle changes for a subject in the second set of subject brains based on the pattern of regional cortical atrophy.

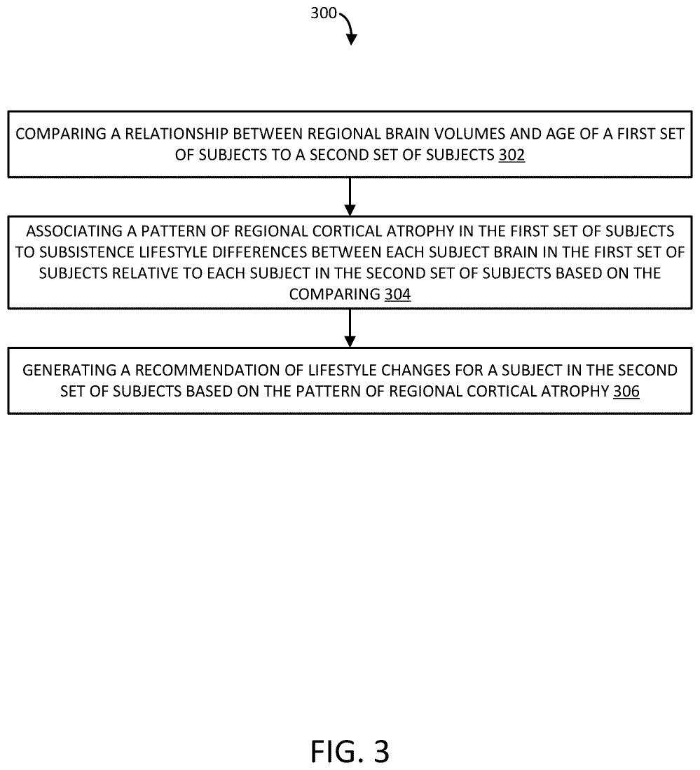

With reference to FIG. 3, a method 300 is provided. The method may include comparing a relationship between regional brain volumes and age of a first set of subjects to a second set of subjects (block 302). The method may include associating a pattern of regional cortical atrophy in the first set of subjects to subsistence lifestyle differences between each subject brain in the first set of subjects relative to each subject in the second set of subjects based on the comparing (block 304). The method may include generating a recommendation of lifestyle changes for a subject in the second set of subjects based on the pattern of regional cortical atrophy (block 306).

One or more further aspect may also be provided. For instance, the method may include normalizing brain volumes from brain images for each subject prior to the comparing, the brain images generated from magnetic resonance imaging (MRI) scans of the brain of each subject. The normalizing may include dividing each regional volume of the subject's brain by a total intracranial volume of the subject's brain to form a set of normalized regional volumes for each subject's brain. The normalizing may include performing a linear regression on the set of normalized regional volumes predicted by age for each subject's brain.

An article of manufacture is also provided. The article of manufacture may include one or more aspects of the aforementioned methods. For instance, the article may include a tangible, non-transitory computer-readable storage medium having instructions stored thereon that, in response to execution by one or more processors, cause the one or more processors to perform operations including various aspects. The various aspects may include receiving, via the one or more processors, a plurality of brain images, the plurality of brain images generated from computed tomography (CT) scans of a brain of each subject. The various aspects may include segmenting, via the one or more processors and through a cortical parcellation scheme, cortical gray matter from the plurality of brain images into gyral and sulcal structures to form a gray matter probability map. The aspects may include correcting, via the one or more processors, a radiodensity gradient along an inferior-superior axis of a computed topography (CT) volume. The aspects may include iterating, via the one or more processors, linear transformations to register the cortical gray matter to each of a subject's gray matter mask. The aspects may include generating, via the one or more processors, an aggregated brain data for each subject's brain including a label for at least a set of voxels in the brain for each subject. The aspects may include normalizing, via the one or more processors, brain volumes from the aggregated brain data for each subject.

Moreover, in various embodiments of the article of manufacture, the normalizing includes dividing each regional volume of the subject's brain by a total intracranial volume of the subject's brain to form a set of normalized regional volumes for each subject's brain and performing a linear regression on the set of normalized regional volumes predicted by age for each subject's brain.

As used herein, a “script” refers to instructions for a computing device to carry out one or more tasks automatically. As used herein, the term “network” includes any cloud, cloud computing system, or electronic communications system or method which incorporates hardware and/or software components. Communication among the parties may be accomplished through any suitable communication channels, such as, for example, a telephone network, an extranet, an intranet, internet, personal internet device, online communications, satellite communications, off-line communications, wireless communications, transponder communications, local area network (LAN), wide area network (WAN), virtual private network (VPN), networked or linked devices, keyboard, mouse, and/or any suitable communication or data input modality. Moreover, although the system may be described herein as being implemented with TCP/IP communications protocols, the system may also be implemented using IPX, APPLETALK®, IPv6, NetBIOS, any tunneling protocol (e.g., IPsec, SSH, etc.), or any number of existing or future protocols. If the network is in the nature of a public network, such as the internet, it may be advantageous to presume the network to be insecure and open to eavesdroppers. Specific information related to the protocols, standards, and application software utilized in connection with the internet is generally known to those skilled in the art and, as such, need not be detailed herein.

“Cloud” or “Cloud computing” or “cloud computing infrastructure” includes a model for enabling convenient, on-demand network access to a shared pool of configurable computing resources (e.g., networks, servers, storage, applications, and services) that can be rapidly provisioned and released with minimal management effort or service provider interaction. Cloud computing may include location-independent computing, whereby shared servers provide resources, software, and data to computers and other devices on demand. Reference to a “device” or processor or memory or the like may include cloud resources, non-cloud resources, or combinations of cloud and non-cloud resources.

Computer programs (also referred to as computer control logic) are stored in main memory and/or secondary memory. Computer programs may also be received via communications interface. These computer program instructions may be loaded onto a general purpose computer, special purpose computer, controller, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer, controller, or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

In various embodiments, software may be stored in a computer program product and loaded into a computer system using a removable storage drive, hard disk drive, or communications interface. The control logic (software), when executed by the processor or controller, causes the processor or controller to perform the functions of various embodiments as described herein. In various embodiments, hardware components may take the form of application specific integrated circuits (ASICs). Implementation of the hardware so as to perform the functions described herein will be apparent to persons skilled in the relevant art(s).

As will be appreciated by one of ordinary skill in the art, the system may be embodied as a customization of an existing system, an add-on product, a processing apparatus executing upgraded software, a stand-alone system, a distributed system, a method, a data processing system, a device for data processing, and/or a computer program product. Accordingly, any portion of the system or a module may take the form of a processing apparatus executing code, an internet based embodiment (e.g., an internet-based driving command system), an entirely hardware embodiment, or an embodiment combining aspects of the internet, software, and hardware. Furthermore, the system may take the form of a computer program product on a computer-readable storage medium having computer-readable program code means embodied in the storage medium. Any suitable computer-readable storage medium may be utilized, including hard disks, solid state storage media, CD-ROM, BLU-RAY DISC®, optical storage devices, magnetic storage devices, and/or the like.

The system and method may be described herein in terms of functional block components, screen shots, optional selections, and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, C#, JAVA®, JAVASCRIPT®, JAVASCRIPT® Object Notation (JSON), VBScript, Macromedia COLD FUSION, COBOL, MICROSOFT® company's Active Server Pages, assembly, PERL®, PHP, awk, PYTHON®, Visual Basic, SQL Stored Procedures, PL/SQL, any UNIX® shell script, and extensible markup language (XML) with the various algorithms being implemented with any combination of data structures, objects, processes, routines or other programming elements. Further, it should be noted that the system may employ any number of techniques for data transmission, signaling, data processing, network control, and the like. Still further, the system could be used to detect or prevent security issues with a client-side scripting language, such as JAVASCRIPT®, VBScript, or the like.

The system and method are described herein with reference to screen shots, block diagrams and flowchart illustrations of methods, apparatus, and computer program products according to various embodiments. It will be understood that each functional block of the block diagrams and the flowchart illustrations, and combinations of functional blocks in the block diagrams and flowchart illustrations, respectively, can be implemented by computer program instructions.

In various embodiments, components, modules, and/or engines of the systems may be implemented as applications or apps. Apps are typically deployed in the context of a mobile operating system, including for example, a WINDOWS® mobile operating system, an ANDROID® operating system, an APPLE® iOS operating system, a BLACKBERRY® company's operating system, and the like. The app may be configured to leverage the resources of the larger operating system and associated hardware via a set of predetermined rules which govern the operations of various operating systems and hardware resources. For example, where an app desires to communicate with a device or network other than the mobile device or mobile operating system, the app may leverage the communication protocol of the operating system and associated device hardware under the predetermined rules of the mobile operating system. Moreover, where the app desires an input from a user, the app may be configured to request a response from the operating system which monitors various hardware components and then communicates a detected input from the hardware to the app.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions. Further, illustrations of the process flows, and the descriptions thereof may make reference to user WINDOWS®/LINUX®/UNIX® applications, webpages, websites, web forms, prompts, etc. Practitioners will appreciate that the illustrated steps described herein may comprise, in any number of configurations, including the use of WINDOWS®/LINUX®/UNIX® applications, webpages, web forms, popup WINDOWS®/LINUX®/UNIX® applications, prompts, and the like. It should be further appreciated that the multiple steps as illustrated and described may be combined into single webpages and/or WINDOWS®/LINUX®/UNIX® applications but have been expanded for the sake of simplicity. In other cases, steps illustrated and described as single process steps may be separated into multiple webpages and/or WINDOWS®/LINUX®/UNIX® applications but have been combined for simplicity.

The computers discussed herein may provide a suitable website or other internet-based graphical user interface (GUI) which is accessible by users. In one embodiment, MICROSOFT® company's Internet Information Services (IIS), Transaction Server (MTS) service, and an SQL SERVER® database, are used in conjunction with MICROSOFT® operating systems, WINDOWS NT® web server software, SQL SERVER® database, and MICROSOFT® Commerce Server. Additionally, components such as ACCESS® software, SQL SERVER® database, ORACLE® software, SYBASE® software, INFORMIX® software, MYSQL® software, INTERBASE® software, etc., may be used to provide an Active Data Object (ADO) compliant database management system. In one embodiment, the APACHE® web server is used in conjunction with a LINUX® operating system, a MYSQL® database, and PHP, Ruby, and/or PYTHON® programming languages.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In re Nuijten to fall outside the scope of patentable subject matter under 35 U.S.C. § 101.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching may be used throughout the figures to denote different parts but not necessarily to denote the same or different materials.

Methods, systems, and articles are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. § 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A method for analyzing age-related regional volumetric change of a brain, the method comprising:
segmenting, via a cortical parcellation scheme, cortical gray matter from a plurality of brain images into gyral and sulcal structures to form a gray matter probability map, the plurality of brain images generated from computed tomography (CT) scans of the brain;
correcting a radiodensity gradient along an inferior-superior axis of a computed topography (CT) volume;
iterating linear transformations to register the cortical gray matter to each of a subject's gray matter mask; and
generating an aggregated brain data for the brain including a label for at least a set of voxels in the brain for each subject according to the cortical parcellation scheme; and
normalizing a brain volume from the aggregated brain data.

2. The method of claim 1, wherein the cortical parcellation scheme is a cortical parcellation scheme.

3. The method of claim 1, further comprising binarizing the gray matter probability map.

4. The method of claim 1, further comprising enhancing registration quality of each of the subject's gray matter mask via non-linear registration.

5. The method of claim 1, wherein the normalizing further comprises dividing each regional volume of the subject's brain by a total intracranial volume of the subject's brain to form a set of normalized regional volumes for each subject's brain.

6. The method of claim 5, wherein the normalizing further comprises performing a linear regression on the set of normalized regional volumes predicted by age for each subject's brain.

7. The method of claim 6, further comprising computing standard regression coefficients by converting the set of normalized regional volumes for each subject's brain to z-scores prior to regression.

8. The method of claim 6, further comprising comparing the set of normalized regional volumes of a first set of subject brains to the set of normalized regional volumes of a second set of subject brains.

9. The method of claim 8, wherein the first set of subject brains and the second set of subject brains are determined based on a geographical location of each subject.

10. The method of claim 8, further comprising calculating a rate of decrease of cortical gray matter volume of the first set of subject brains and the second set of subject brains.

11. The method of claim 10, wherein the comparing further comprises comparing the rate of decrease of cortical gray matter volume of the first set of subject brains to the second set of subject brains.

12. The method of claim 8, wherein the comparing further comprises comparing a portion of the first set of subject brains with a portion of the second set of subject brains based on a sex of each subject.

13. The method of claim 8, associating a pattern of regional cortical atrophy in the first set of subject brains to subsistence lifestyle differences between each subject from the first set of subject brains relative to each subject in the second set of subject brains based on the comparing.

14. The method of claim 13, further comprising generating a recommendation of lifestyle changes for a subject in the second set of subject brains based on the pattern of regional cortical atrophy.

15. A method, comprising:
comparing a relationship between regional brain volumes and age of a first set of subjects to a second set of subjects;
associating a pattern of regional cortical atrophy in the first set of subjects to subsistence lifestyle differences between each subject brain in the first set of subjects relative to each subject in the second set of subjects based on the comparing; and
generating a recommendation of lifestyle changes for a subject in the second set of subjects based on the pattern of regional cortical atrophy.

16. The method of claim 15, further comprising normalizing brain volumes from brain images for each subject prior to the comparing, the brain images generated from CT scans of the brain of each subject.

17. The method of claim 16, wherein the normalizing further comprises dividing each regional volume of the subject's brain by a total intracranial volume of the subject's brain to form a set of normalized regional volumes for each subject's brain.

18. The method of claim 17, wherein the normalizing further comprises performing a linear regression on the set of normalized regional volumes predicted by age for each subject's brain.

19. An article of manufacture including a tangible, non-transitory computer-readable storage medium having instructions stored thereon that, in response to execution by one or more processors, cause the one or more processors to perform operations comprising:
receiving, via the one or more processors, a plurality of brain images, the plurality of brain images generated from CT scans of a brain of each subject;
segmenting, via the one or more processors and through a cortical parcellation scheme, cortical gray matter from the plurality of brain images into gyral and sulcal structures to form a gray matter probability map;
correcting, via the one or more processors, a radiodensity gradient along an inferior-superior axis of a computed topography (CT) volume;
iterating, via the one or more processors, linear transformations to register the cortical gray matter to each of a subject's gray matter mask;
generating, via the one or more processors, an aggregated brain data for each subject's brain including a label for at least a set of voxels in the brain for each subject; and
normalizing, via the one or more processors, brain volumes from the aggregated brain data for each subject.

20. The article of manufacture of claim 19, wherein the normalizing further comprises:
dividing each regional volume of the subject's brain by a total intracranial volume of the subject's brain to form a set of normalized regional volumes for each subject's brain; and
performing a linear regression on the set of normalized regional volumes predicted by age for each subject's brain.
